(19) United States
(12) Patent Application Publication
(10) Pub. No.: US 2021/0178086 A1
(43) Pub. Date: Jun. 17, 2021

(54) APPARATUS FOR INJECTING INFUSION SOLUTION

(71) Applicant: Industry-Academic Cooperation Foundation, Chosun University, Gwangju (KR)

(72) Inventors: Ji Yun CHOI, Gwangju (KR); Jung Ah KIM, Gwangju (KR)

(73) Assignee: Industry-Academic Cooperation Foundation, Chosun University, Gwangju (KR)

(21) Appl. No.: 17/115,414

(22) Filed: Dec. 8, 2020

(30) Foreign Application Priority Data
Dec. 17, 2019 (KR) 10-2019-0169119

(51) Int. Cl. A61M 5/44; A61M 5/168; A61M 5/148; G16H 20/17

(52) U.S. Cl. CPC A61M 5/445; A61M 5/16881; A61M 5/148; G16H 20/17; A61M 2205/583; A61M 2205/3327; A61M 2205/3337; A61M 2205/18; A61M 2205/8206

(57) ABSTRACT

An apparatus for injecting an infusion solution is provided, and the apparatus includes a housing including a receiving part to receive an infusion solution bag in the receiving part and an infusion solution tube connected with the infusion solution bag and withdrawn, and a pressing member received in the receiving part to self-inflate as air is supplied and to press the infusion solution bag.

### CROSS-REFERENCE TO RELATED APPLICATIONS

A claim for priority under 35 U.S.C. § 119 is made to Korean Patent Application No. 10-2019-0169119 filed on Dec. 17, 2019 in the Korean Intellectual Property Office, the entire contents of which are hereby incorporated by reference.

### BACKGROUND

Embodiments of the inventive concept described herein relate to an apparatus for injecting an infusion solution.

In general, an infusion solution is injected in surgery or hospitalization. The infusion solution, which is an infusion solution injected into a human body when shock, dehydration, or malnutrition occurs in the human body, is a kind of drug containing various electrolyte solutions such as blood, physiological saline the same osmotic pressure as that of the blood, or glucose.

Conventionally, to supply the infusion solution, an infusion solution bag is placed in a higher position than the human body by hanging upside down on an infusion solution pole, and the infusion solution is dropped by gravity to be injected into the human body through an infusion solution tube connected with the infusion solution bag, an injection tube, and an injection needle.

However, conventionally, the infusion solution has to be injected by dragging the infusion solution pole in the state that the infusion solution bag is hung upside down on the infusion solution pole. Accordingly, a patient may feel inconvenient when moving.

In other words, when the patient moves to take a walk or receive an examination, the patient has to move with the infusion solution pole. The patient may feel inconvenient when moving. In addition, the patient is restricted in moving when crossing over a step on the floor or going upstairs.

Further, conventionally, since an amount of an infusion solution injected into the human body is not recognized, the amount of infusion solution has to be frequently checked. In addition, after the infusion solution is completely injected, the blood may flow back, which requires an attention.

### SUMMARY

Embodiments of the inventive concept provide an apparatus for injecting an infusion solution, capable of injecting the infusion solution without a behavioral restriction in movement and regardless of a place.

Embodiments of the inventive concept provide an apparatus for injecting an infusion solution, capable of easily managing and injecting the infusion solution in a remote place.

According to an exemplary embodiment, an apparatus for injecting an infusion solution may include a housing including a receiving part to receive an infusion solution bag in the receiving part and an infusion solution tube connected with the infusion solution bag and withdrawn, and a pressing member received in the receiving part to self-inflate as air is supplied, to press the infusion solution bag.

In this case, the apparatus may further include an air valve provided in the housing to introduce or withdraw the air into the receiving part.

The apparatus may include an air bag received in the receiving part to press the infusion solution bag.

The pressing member may include an automatic-charging mat.

The apparatus may further include a heating cable received in the receiving part to heat the infusion solution bag to be at a constant temperature, and a battery to supply power to the heating cable.

The apparatus may further include a power switch to turn on or turn off power supplied to the heating cable.

The apparatus may further include an infusion solution sensing unit to sense an amount of an infusion solution discharged from the infusion solution bag, and an infusion solution regulating unit to regulate an amount of the infusion solution discharged from the infusion solution bag, based on infusion solution data sensed by the infusion solution sensing unit.

The apparatus may further include a communication module to transmit the infusion solution data sensed by the infusion solution sensing unit to an external device. An opening degree of the infusion solution regulating unit may be adjusted in response to a control signal received through the communication module.

The apparatus may further include an alarm generating unit to notify an amount of an infusion solution remaining in the infusion solution bag around.

The apparatus may further include a display unit provided in the housing to display information associated with the infusion solution.

The apparatus may further include a checking window provided in the housing such that a flow of an infusion solution flowing through the infusion solution tube checks with naked eyes of a user.

As described above, according to the inventive concept, the infusion solution may be injected without the behavioral restriction and regardless of the place.

In addition, the infusion solution may be easily managed even in the remote place.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating an apparatus for injecting an infusion solution, according to an embodiment of the inventive concept;

FIG. 2 is a sectional view taken along line A-A of FIG. 1;

FIG. 3 is a sectional view taken along line B-B of FIG. 1;

FIG. 4 is a view illustrating the apparatus for injecting the infusion solution of FIG. 1, which is received in a backpack, according to an embodiment; and

FIG. 5 is a backpack to receive the apparatus for injecting the infusion solution of FIG. 1, according to another embodiment.

### DETAILED DESCRIPTION

Advantage points and features of the inventive concept and a method of accomplishing thereof will become apparent from the following description with reference to the following drawings, wherein embodiments will be described in detail with reference to the accompanying drawings. However, the inventive concept may be embodied in various different forms, and should not be construed as being limited only to the illustrated embodiments. Rather, these embodiments are provided as examples so that the inventive concept will be thorough and complete, and will allow those skilled in the art to fully understand the scope of the inventive concept. The inventive concept may be defined by scope of the claims.

The terminology used herein is provided for explaining embodiments, but the inventive concept is not limited thereto. As used herein, the singular terms “a” “an” and “the” are intended to include the plural forms as well, unless the context clearly indicates otherwise. Furthermore, it will be further understood that the terms “comprises”, “comprising,” “includes” and/or “including”, when used herein, specify the presence of stated elements, steps, operations, and/or devices, but do not preclude the presence or addition of one or more other components, steps, operations and/or devices. The same reference numerals will be assigned to the same component throughout the whole specification, and “and/or” refers to that components described include not only individual components, but at least one combination of the components. It will be understood that, although the terms “first”, “second”, etc., may be used herein to describe various components, these components should not be limited by these terms. These terms are only used to distinguish one component from another component. Thus, a first component to be described below may be a second component without departing from the teachings of the inventive concept.

Unless otherwise defined, all terms (including technical and scientific terms) used herein have the same meaning as commonly understood by those skilled in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, embodiments of the inventive concept will be described with reference to accompanying drawings.

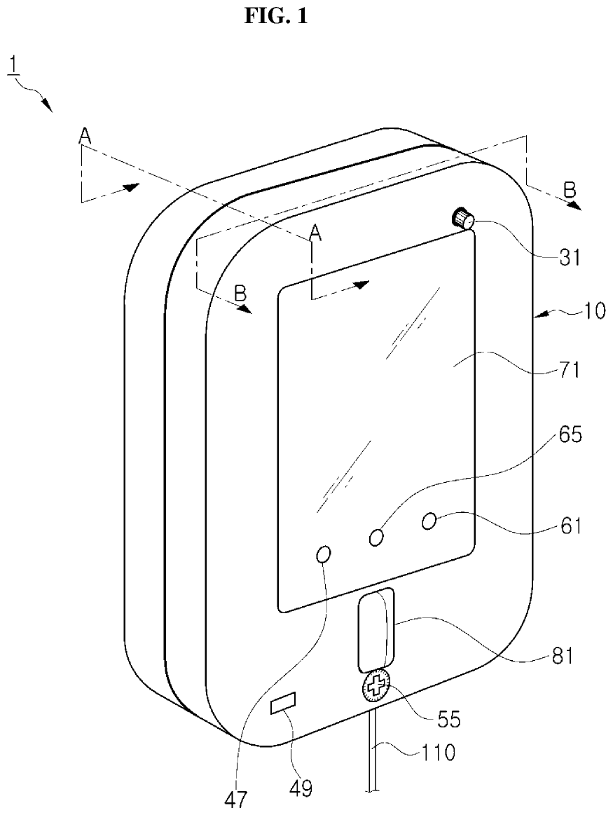

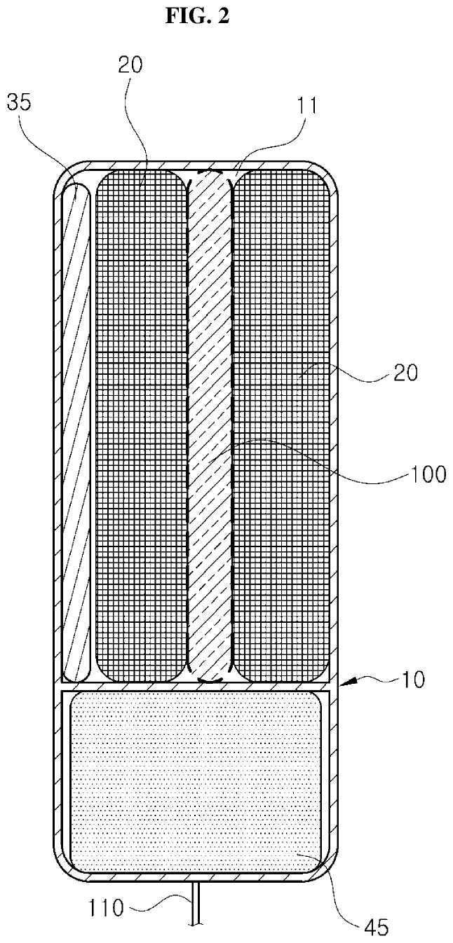

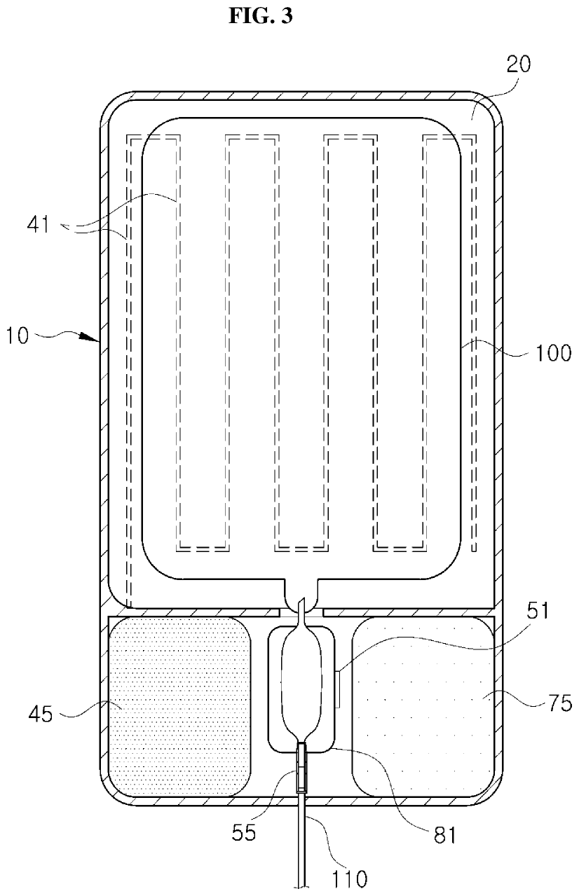

FIGS. 1 to 3 illustrate an apparatus for injecting an infusion solution, according to an embodiment of the inventive concept.

As in the drawings, according to an embodiment of the inventive concept, an apparatus 1 for injecting the infusion solution may include a housing 10 and a pressing member 20.

The housing 10 has a hollowed structure, and has a receiving part 11 formed therein.

The housing 10 is provided in the form divided into a left part and a right part, which are coupled to each other through a coupling unit such as a zipper.

The housing 10 may include a material having a higher hardness such that the housing 10 is prevented from being elastically deformed when pressed by the pressing member 20. Accordingly, when self-inflating, the pressing member 20 inflates toward an infusion solution bag 100 instead of the housing 10 to compress the infusion solution bag 100.

The infusion solution bag 100 is received in the receiving part 11. The infusion solution bag 100 received in the receiving part 11 is connected with an infusion solution tube 110, and the infusion solution tube 110 is provided to be withdrawn from the housing 10.

The pressing member 20 is received in the receiving part 11 while surrounding the infusion solution bag 100.

The pressing member 20 is manufactured using a material, which self-inflates as air is supplied, to press the infusion solution bag 100.

For example, when external air is supplied into the receiving part 11, the volume of the pressing member 20 is increased to press the infusion solution bag 100 surrounded by the pressing member 20. Accordingly, the infusion solution contained in the infusion solution bag 100 is discharged from the housing 10 through the infusion solution tube 110 through pressure applied by the pressing member 20 instead of gravity.

To the contrary, when air is discharged from the receiving part 11, the volume of the pressing member 20 is decreased, and the infusion solution bag 100 surrounded by the pressing member 20 is not pressed any more. Accordingly, the infusion solution contained in the infusion solution bag 100 is not pressed by the pressing member 20 and not discharged from the housing 10 through the infusion solution tube 110. Accordingly, in the state that the air is discharged from the receiving part 11, the infusion solution bag 100 received in the receiving part 11 may be simply replaced with new one.

As described above, a battery is not required to press the infusion solution bag 100 as the pressing member 20 self-inflates. In addition, according to an embodiment of the inventive concept, even if the apparatus 1 for injecting the infusion solution is not positioned higher than a human body, the infusion solution in the infusion solution bag 100 may be smoothly injected into a blood vessel through pressure applied by the pressing member 20.

In this case, the pressing member 20 may include a typical automatic-charging mat. In addition, although not illustrated, a part between the pressing member 20 and the infusion solution bag 100 may include a board to press the infusion solution bag 100 without being elastically deformed when the pressing member 20 applies pressure.

In addition, an air valve 31 is provided in the housing 10 to introduce or withdraw air. In this case, the air valve 31 is preferably a one-way valve. For example, the air valve 31 may have a structure in which external air is automatically introduced into the receiving part 11 when the air valve 31 is open.

An air bag 35 is provided in the housing 10 to press the infusion solution bag 100.

The air bag 35 is secondarily used when rapidly injecting the infusion solution or when the pressing member 20 applies less pressure. In other words, the air bag 35 is manually pressed to press the infusion solution bag 100 together with the pressing member 20, thereby rapidly discharging the infusion solution from the infusion solution bag 100 through the infusion solution tube 110.

In addition, according to an embodiment of the inventive concept, the apparatus 1 for injecting the infusion solution may further include a heating cable 41.

The heating cable 41 is received in the receiving part 11 and arranged along a board surface of the pressing member 20 to heat the infusion solution bag 100 to be at a constant temperature.

The heating cable 41 is supplied with power from a battery 45 provided at a lower portion of the receiving part 11 in the housing 10.

The power supplied from the battery 45 to the heating cable 41 is turned on or off by a power switch 47 provided outside the housing 10.

The housing 10 is provided at one side thereof with a charging terminal 49 to charge the battery 45.

As described above, as the heating cable 41 is provided, the infusion solution contained in the infusion solution bag 100 may be maintained warmed at a proper temperature to be injected into even a patient sensitive to a temperature without resistance.

In addition, according to an embodiment of the inventive concept, the apparatus 1 for injecting the infusion solution may include an infusion solution sensing unit 51 and an infusion solution regulating unit 55.

The infusion solution sensing unit 51 may sense an amount of infusion solution discharged from the infusion solution bag 100.

According to an embodiment, the infusion solution sensing unit 51 counts the number of drops of the infusion solution, which is discharged through the infusion solution tube 110, per time and calculates an amount of the infusion solution. The number of drops of the infusion solution may be observed by a manager with naked eyes such that the amount of the infusion solution may be calculated. According to another embodiment, the infusion solution sensing unit 51 may include a sensor provided at a portion, to which the infusion solution is dropped, to calculate the amount of the infusion solution.

The infusion solution regulating unit 55 is provided in the infusion solution tube 110 to regulate an amount of an infusion solution discharged from the infusion solution bag 100 based on infusion solution data sensed by the infusion solution sensing unit 51.

The infusion solution regulating unit 55 regulates an amount of the infusion solution to be discharged through the infusion solution tube 110 by adjusting an opening degree of the infusion solution tube 110. The infusion solution regulating unit 55 may be manually operated or automatically operated using a motor.

In addition, the infusion solution data sensed by the infusion solution sensing unit 51 may be transmitted to an external device such as a smartphone, or a management server (not illustrated) through a communication module 61. Accordingly, a patient manager, such as a doctor and a nurse, may easily manage an infusion solution to be injected into a patient even in a remote place.

In addition, the patient manager may simply detect an amount of an infusion solution injected based on the infusion solution data transmitted through the communication module 61, and may transmit a control signal to the infusion solution regulating unit 55 through the communication module 61 to adjust the opening degree of the infusion solution regulating unit 55, such that the infusion solution is adjusted to be in a desired amount and injected.

Further, according to an embodiment of the inventive concept, the apparatus 1 for injecting the infusion solution may further include an alarm generating unit 65.

The alarm generating unit 65 may notify an amount of an infusion solution remaining in the infusion solution bag 100 around by outputting a sound through a speaker provided in the housing 10 or by emitting light through a lamp provided in a display unit 71 to be described later.

For example, when 50% of the infusion solution remains in the infusion solution bag 100, an alarm sound intermittently rings through the speaker or green light may be intermittently emitted, for a specified period of time. When 10% of the infusion solution remains in infusion solution bag 100, an alarm sound may continuously ring through the speaker, or red light may be emitted, for a specified period of time.

Meanwhile, according to an embodiment of the inventive concept, the apparatus 1 for injecting the infusion solution may further include the display unit 71.

The display unit 71 is provided at one side of the housing 10 to be exposed to display information associated with the infusion solution.

The display unit 71 may display an alarm message, a call message, a thermal message, a message of an amount of an infusion solution injected and an amount of the infusion solution remaining, a time to replace the infusion solution bag 100, or other messages. The display unit 71 may include a touch screen.

In this case, although the display unit 71 uses power supplied from the battery 45, the display unit 71 may use power supplied from an auxiliary battery 75 as the auxiliary battery 75 is additionally and separately provided. The auxiliary battery 75 may be charged through the charging terminal 49 provided at one side of the housing 10.

In addition, a checking window 81 is provided in the housing 10 such that the flow of an infusion solution through the infusion solution tube 110 is checked with naked eyes.

The checking window 81 is manufactured using a transparent material provided on a path of the infusion solution tube 110 such that the infusion solution tube 110 is easily checked from the outside.

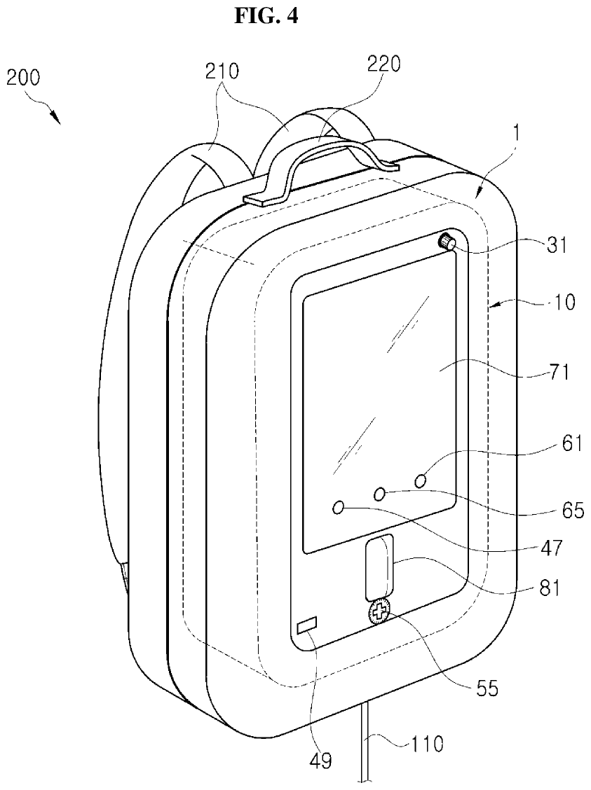

Meanwhile, FIG. 4 is a view illustrating the apparatus 1 for injecting the infusion solution, which is received in a backpack 200, according to an embodiment of the inventive concept.

As illustrated in FIG. 4, according to an embodiment of the inventive concept, the apparatus 1 for injecting the infusion solution may be received and used in the backpack 200 provided to expose the display unit 71.

The backpack 200 may include a band 210 such that a user carries the backpack 200 on the shoulder of the user.

Accordingly, according to an embodiment of the inventive concept, a patient may freely move with the apparatus 1 for injecting the infusion solution on the shoulder of the user regardless of a place while receiving the infusion solution.

In addition, the backpack 200 has an auxiliary band 220 in a ring shape such that the patient may use the backpack 200 by hanging the backpack 200 on a structure.

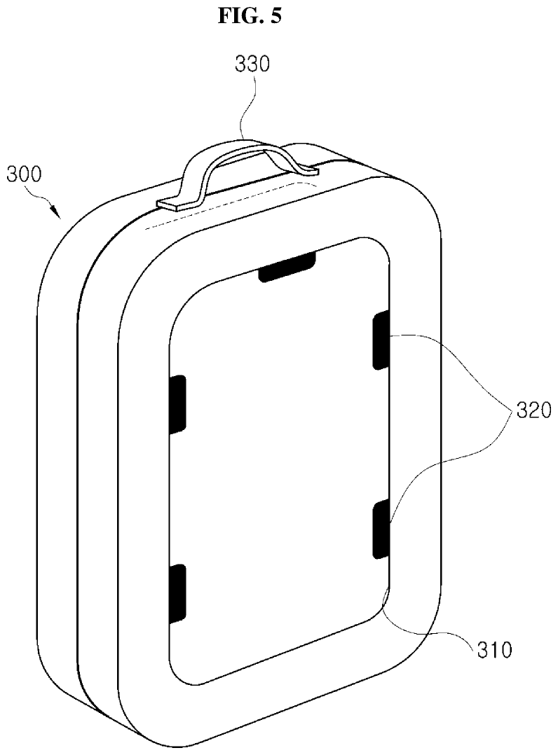

FIG. 5 is a backpack to receive an apparatus for injecting an infusion solution, for use, according to another embodiment of the inventive concept.

A backpack 300 is formed in one side thereof with an opening 310 such that a display unit of the apparatus for injecting the infusion solution is exposed to the outside.

In addition, a plurality of fixing clips 320 are provided at a circumference of the opening 310 of the backpack 300 to fix the display unit of the apparatus for injecting the infusion solution.

In addition, a handle 330 is provided at an upper portion of the backpack 300 such that the backpack 300 having the apparatus for injecting the infusion solution is carried or placed in a hospital room.

As described above, according to an embodiment of the inventive concept, the apparatus for injecting the infusion solution may be received and used in variously-shaped backpacks.

In a backpack having no display unit exposed, data on an amount of an infusion solution contained in an infusion solution pack may be transmitted to a user terminal, such as a smartphone, through a communication module wiredly or wirelessly.

As described above, according to the inventive concept, the infusion solution is not supplied by gravity, but supplied by pressing the infusion solution bag 100 by means of the pressing member 20 self-inflating as air is supplied. In addition, the apparatus for injecting the infusion solution is easily carried and represents excellent mobility. Accordingly, the patient has no behavioral restriction in movement and may receive the infusion solution regardless of a place.

Meanwhile, although the previous embodiment has been described in that the battery 45 and the auxiliary battery 75 are charged with power through the charging terminal 49, the inventive concept is not limited thereto. The battery 45 and the auxiliary battery 75 may be mounted and charged on a separate wireless charger.

In addition, according to an embodiment of the inventive concept, the apparatus 1 for injecting the infusion solution may be received and used in a backpack in a specific character shape.

Accordingly, the resistance of children against the injection of an infusion solution may be reduced, so the children having the apparatus 1 for injecting the infusion solution on their shoulders may receive the infusion solution while freely moving regardless of a place.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. An apparatus for injecting an infusion solution, the apparatus comprising:
a housing including a receiving part to receive an infusion solution bag in the receiving part and an infusion solution tube connected with the infusion solution bag and withdrawn; and
a pressing member received in the receiving part and configured to self-inflate as air is supplied and to press the infusion solution bag.

2. The apparatus of claim 1, further comprising:
an air valve provided in the housing to introduce or withdraw the air into the receiving part.

3. The apparatus of claim 1, further comprising:
an air bag received in the receiving part to press the infusion solution bag.

4. The apparatus of claim 1, wherein the pressing member includes an automatic-charging mat.

5. The apparatus of claim 1, further comprising:
a heating cable received in the receiving part to heat the infusion solution bag to be at a constant temperature; and
a battery configured to supply power to the heating cable.

6. The apparatus of claim 5, further comprising:
a power switch to turn on or turn off the power supplied to the heating cable.

7. The apparatus of claim 1, further comprising:
an infusion solution sensing unit configured to sense an amount of an infusion solution discharged from the infusion solution bag; and
an infusion solution regulating unit configured to regulate an amount of the infusion solution discharged from the infusion solution bag, based on infusion solution data sensed by the infusion solution sensing unit.

8. The apparatus of claim 7, further comprising:
a communication module configured to transmit the infusion solution data sensed by the infusion solution sensing unit to an external device,
wherein an opening degree of the infusion solution regulating unit is adjusted in response to a control signal received through the communication module.

9. The apparatus of claim 7, further comprising:
an alarm generating unit configured to notify an amount of an infusion solution remaining in the infusion solution bag around.

10. The apparatus of claim 1, further comprising:
a display unit provided in the housing to display information associated with the infusion solution.

11. The apparatus of claim 1, further comprising:
a checking window provided in the housing such that a flow of an infusion solution through the infusion solution tube checks with naked eyes of a user.
